(19) United States
(12) Patent Application Publication
(10) Pub. No.: US 2014/0267116 A1
(43) Pub. Date: Sep. 18, 2014

(54) Finger Splint System

(71) Applicant: Matthew A. Weiner, Ambler, PA (US)

(72) Inventor: Matthew A. Weiner, Ambler, PA (US)

(21) Appl. No.: 14/211,787

(22) Filed: Mar. 14, 2014

Related U.S. Application Data
(60) Provisional application No. 61/782,536, filed on Mar. 14, 2013.

(51) Int. Cl. A61F 5/058; G06F 3/041

(52) U.S. Cl. CPC A61F 5/05866; G06F 3/0416
USPC 345/173; 602/22

(57) ABSTRACT

A splint system used to interface with a touchscreen, where the splint system comprises a housing having a base with ends and sides, at least one circumferentially adjustable structure contiguous with the base extending from the sides to a longitudinal axis of the base, and an electromechanical device embodied in the base. One end of the base has an upward curve extending therefrom.

### BACKGROUND OF THE INVENTION

The invention relates to a splint system. In particular, the invention relates to a splint that interacts with an electronic device.

Injuries to the finger may require a splint for healing those injuries, such as fractures, inflammatory processes, sprains, strains, lacerations, soft tissue tears and repetitive use injuries. Splints are typically designed to hold fingers to be straight or bent into a curved position such that they support, compress, and protect the finger. Foam padding or a dressing may be added to allow air to circulate, aiding in healing and improving wearer comfort. Splints may also be used to selectively support and protect selected ligaments or soft tissue structures around a joint. A splint may be a supportive or protective apparatus that aids in initiation and performance of motion by the supported or adjacent parts.

In current use, splints may be secured to the finger with adhesive tape, hook and loop closure or ties wrapped around the base of the splint. These methods of fixation may be difficult to apply for older or disabled users. These methods of fixation are also cumbersome to apply for intermittent protection during performance of activities of daily living. Many splints are made of foam padding and hook and loop closures that become wet with daily living activities. Wet materials may cause skin maceration, skin breakdown and unhygienic conditions. Because the splint prevents normal movement of the finger, it may be uncomfortable for the wearer. Wearing a splint may also encumber one's everyday movements and tasks on computer devices, such as typing and using touch screen devices. The interface surface of touchscreen devices are often made of Gorilla Glass™ and other screen materials that are exceptionally hard and non-resilient materials that do not attenuate shock like the keys on a desktop keyboard. Repetitive use of typing, tapping, swiping and pinching may place the user at risk for repetitive stress injuries and aggravate previously injured fingers or arthritic fingers. Newer tablet keyboards have also become very thin (about 3-4 mm) and may not have any spring action in the keys. It is burdensome for a user to constantly pick up a stylus to interact with a touchscreen while inputting data. Additionally, it is burdensome for the user to utilize a bulky, awkward housing for an electronic device that frequently falls off of the distal finger during use.

All of these problems are both inconvenient and obstructive. Therefore, it is desirable to provide a device that helps to reduce these problems and allows wearers to fully participate in their daily living, work and recreational activities while protecting the finger.

### SUMMARY OF THE INVENTION

One aspect of the invention provides a splint system comprising a housing having a base with ends and sides and at least one circumferentially adjustable structure contiguous with the base extending from the sides to a longitudinal axis of the base for fixing the housing to at least one finger.

Another aspect of the invention provides a splint system comprising a housing having a base with ends and sides and at least one circumferentially adjustable structure contiguous with the base extending from the sides to a longitudinal axis of the base for fixing the housing to a palmar surface of any sized finger, an upward curve extending from one end of the base, and an electromechanical device embodied within the housing.

Yet another aspect of the invention provides a method interfacing with a touchscreen comprising providing a splint system with a housing having a base with ends and sides, at least one circumferentially adjustable structure contiguous with the base extending from the sides to a longitudinal axis of the base, and an electromechanical device embodied in the base, one end of the base having an upward curve extending therefrom; engaging the circumferentially adjustable structures with a portion the at least one finger; positioning the upward curve near a tip of the at least one finger; and activating the electromagnetic device.

### BRIEF DESCRIPTION OF THE DRAWINGS

Various embodiments are best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 shows a side view of one embodiment of the splint system on a finger;

FIG. 2 shows a side frontal view of the splint system of FIG. 1;

FIG. 3 shows a side frontal view of another embodiment of the splint system;

FIG. 4 shows a cross sectional view of one embodiment of a touchscreen, finger anatomy and tine element;

FIG. 5 shows a cross sectional view of another embodiment of a touchscreen, finger anatomy and tine element;

FIG. 6 shows a frontal view of one embodiment of tines through a transparent touchscreen;

FIG. 7 shows an inferior view of another embodiment of tines and a cross section of a touchscreen;

FIG. 8 shows another cross sectional view of a touchscreen, a tine element and finger anatomy;

FIG. 9 shows a side frontal view of another embodiment of a splint system;

FIG. 9a shows a close-up view of one embodiment of an outer surface of an upward curve;

FIG. 10 shows aside frontal view of another embodiment of the splint system;

FIG. 10a shows a close-up view of another embodiment of the outer surface of an upward curve; and

FIG. 11 shows a top view of an embodiment of a 3D surface.

### DETAILED DESCRIPTION OF THE INVENTION

The invention provides a splint system that can be used to prevent or heal an injury of any sized finger(s). The splint system is both easy to wear and comfortable and may be adjustable or be fit for various sized fingers and hands.

FIG. 1 shows one embodiment of the splint system (1) with a housing (2) fixed over a finger (14) with anatomical components. The housing (2) comprises a length suitable to fit the anthropomorphic length of a finger (14) with an upward curve (3) to meet the palmar surface (14a) of the finger (14). The base (4) of the housing (2) has ends and sides and extends along the palmar surface (14a) of the distal phalange (5) and soft tissues of the palmar surface (14a) of the finger (14). Preferably, the base (4) comprises a flat surface, but can be curved to fit the finger (14). The base (4) may have a smooth or a textured surface. The base (4) forms an upward curve (3) to just below the nail bed area. The base (4) and the upward curve (3) may provide protection to the finger (14), including protecting a densely innervated finger pad (14b), which is the subcutaneous fat that constitutes the tips of the fingers, and fingertip (14c). Proximal wings (6) and distal wings (7), or winged structures, rise at an angle from the base (4) to wrap around an edge of the distal phalange (8). The proximal wings (6) extend across a span of the distal interphalangeal joint (9). The proximal wings (6) provide support to the joint (9) during varus and valgus forces on the joint (9) and on the collateral ligament structures (10). The proximal wings (6) also fix themselves along the edge of the distal phalange (8) to provide a degree of fixation for the housing (2) when axial forces act to pull or twist the splint housing (2) off of the finger.

In one embodiment, the proximal wings (6) and distal wings (7) form a cone shape which cams the distal phalange (8), which is also cone-shaped, between the sets of wings (6) and (7) and the base (4) of the splint housing (2). The proximal wings (6) and distal wings (7) are circumferentially adjustable structures (24). These wings (6) and (7) adjust to the soft tissue expansion and contraction as the finger (14) is moved in activities of daily living providing a means of fixation to the finger (14). The two sets of winged structures (6) and (7) provide functional balance to the splint housing (2) to prevent pivoting, twisting, axial disengagement and rotation on the finger (14). In one embodiment, the two sets of wings (6) and (7) contribute with four points of frictional securing contact along the finger (14) to increase the functional stability of the housing (2). A hyperextension force on the housing (2) may be balanced by the winged structures (6) and (7) arranged at about a 60 to 80 degree angle. A flexion force on the distal housing (2) is balanced by the proximal wings (6).

Preferably, the housing (2) and the winged structures (6) and (7) are oval shaped in the frontal plane. The oval shape positions the finger (14) centrally within the base (4) providing maximal surface contact area with the conical ovular shaped housing (2). The proximal wings (6) provide a counterbalance to a hyperextension force on the distal interphalangeal joint (9), thus protecting the volar plate and the palmer soft tissue of the distal interphalangeal joint (9). The distal set of wings (7) provides a counterbalance to a palmer directed force on the upward curve (3). Both sets of wings (6) and (7) and the splint base (4) act to stabilize the distal phalangeal joint (9) during work activities that require various angles of distal interphalangeal joint flexion. The splint housing (2) with the winged structures (6) and (7), lateral base (4) and upward curve (3) take the focally directed forces at the upward curve (3) and distribute them more generally throughout the splint housing (2) to more sturdy proximal anatomical structures of the finger (14).

The splint system (1) may have a functional negative space, or void, (11) as a result of the straight pull molding technique. One half of the mold pushes through the negative space (11) to create a wing shaped cavity in which an injection-molded material may flow. The splint system (1) may also have an electromagnetic device (12) embodied in the base (4) of the housing (2). This splint housing (2) takes a protective ergonomic approach by spanning the whole distal interphalangeal joint (9) while using a conical formation to provide better fixation of the housing (2) to the finger (14) during occupational and leisure activities. There will be some variety of fit of the splint system (1) due to individual finger length. However, even if wings (6) and (7) do not fully span the distal interphalangeal joint (9), a dampening quality will still be appreciated by the user due to the vibration absorption quality of the material of the splint system (1).

In one embodiment, the splint system (1) is configured to fit 1-5 digits of the hand. In another embodiment, the joint angle of the splint (1) is of an angle suitable for healing of an injury about 0-25 degrees. In another embodiment, the housing (2) is in an arcuate form of an angle. The splint (1) may be custom made or manufactured in a number of sizes to best accommodate the variety of finger circumferences, lengths, widths and injury type.

As shown in FIG. 2, the housing (2) may be altered to fit the anatomy of the thumb. In this embodiment, the proximal winged structures (6) are removed, shortened or reshaped to provide an anthropomorphic fit of the housing (2) on the distal phalange of the thumb. This embodiment uses the distal winged structure (7) to secure the housing (2) to the palmer thumb. This form, while sufficient to hold the housing (2) to the thumb, loses the added function by the proximal winged structure (6) to support and protect the distal phalangeal joint (9). In other various embodiments, the splint system (1) may be of a monopartite or multipartite form. In a multipartite splint system (1), sections of the housing (2) may be connected together by a track system, a rail system, a gear system, a hinge system, or a spring system or the splint system (1) may be fabricated with the post production process of ultrasonic welding. In one embodiment, the upward curve (3) is adjustable on a telescoping base (4) to be modified according to a user's finger length.

The base (4) has a longitudinal axis (27), which bisects the base (4). The wings (6) and (7) are contiguous with the base (4) and arise symmetrically to engage the distal phalange (8) and distal phalangeal joint (9). Based on the flex, shape, width and thickness, the wings (6) and (7) may be in a range from soft to stiff. The wings (6) and (7) will contribute to the level of support the splint system (1) will provide the user.

In one embodiment, the splint system (1) has an electromechanical device (EMD) (12). An EMD (12) may be large or small, even on a nanoscale. The EMD (12) device may be a microelectromechanical system (MEMS) that is made up of components between 1 to 100 micrometers in size (i.e., 0.001 to 0.1 mm), and MEMS devices generally range in size from 20 micrometers (20 millionths of a meter) to a millimeter (i.e. 0.02 to 1.0 mm). A MEM has a central unit that processes data (the microprocessor) and several components that interact with the surroundings such as microsensors. The EMD (12) may also be a NEMS or nanoelectromechanical system that is of a class of devices integrating electrical and mechanical functionality on the nanoscale. An EMD (12) may be embodied in the base (4) of the splint under the area that the distal palmar finger would be positioned. The conical formation of the two sets of wings (6) and (7) have an increasing circumference from the distal wing formation (7) to the proximal wing formation (6). On the base (4) of the longitudinal surface of the housing (2) is a negative space (11). The negative space (11) is created through the straight pull molding process. A preferential, cost effective, mass produced method of fabrication is through the straight pull molding process. The negative space (11) is advantageous in its ability to provide airflow to the palmar surface (14a) of the finger (14) and allow perspiration to evaporate during use. The negative space (11) can also be a functional negative space (11) to allow the embodiment of the EMD (12) to be within the splint housing (2). Additionally, the negative space (11) may be expanded and enlarged distally to include the EMD (12). The functional negative space (11) may act as a drop-in mount for the EMD (12). The EMD (12) may be additionally secured by the palmer surface of the finger (14). The drop-in mount may have molded features that allow the EMD (12) to click into place. The negative space (11) may also work in conjunction with the base (4) under the fingertip (14c), including the upward curve (3), to position an EMD (12). In one embodiment, the EMD (12) may be fixated to the housing (2) by a molded mounting system, male/female connective elements or other means of fixation either under the base (4) over the dorsal finger. The EMD (12) may also be attached to the housing (2) using an adhesive material. A negative space (11) taking the geometric form of the EMD (12) may be molded into the base (4) to allow the EMD (12) to embody the housing (2) under the finger pad (14b) of the distal phalange (5). The depth of the base (4) may be modified to allow the embodiment of an EMD (12). The base (4) may have features like a hole (not shown) to allow communication of an externally fixated EMD (12) to a sensor or actuator electrode under the palmar portion of the finger (14). The splint system (1) with one or two sets of wings (6) and (7) is preferably manufactured with the straight pull molding technique or with all current or future 3D printing processes.

For an EMD (12) to embody the splint system (1), the housing (2) may have a pocket, feature, sleeve, removable tray, tube or other geometric negative space (11) in which to fit. Preferably, the EMD (12) has biological, chemical, thermal, light, mechanical, and/or electromagnetic functions. The EMD (12) may be a biomedical/chemical sensor or actuator device, rechargeable or non-rechargeable power source, microcomputer CPU, micro memory device, microphone, micro digital camera, micro speaker, micro drug delivery device, microscanner device, a micro ultrasound device, micro computer interface device, micro wireless transmission device, micro blood assay device, microarray, gyroscope, accelerometer, micro LED display device, micro LED, fingerprint reader, haptic device, micro toggle switch, micro-optical sensing device and/or micro-laser. The housing (2) may be used to house an artificial fingerprint technology to allow a user to have a unique identifier to be read by a scanner or computer system. In one embodiment, a quick response code may be applied to the housing (2) to identify the user.

In one embodiment, the splint system (1) has a monopartite housing (2) with a base (4) and an EMD (12) with a sensor or actuator under the distal palmar surface (14a) of the finger (14). The fingertip contact area under the distal base (4) and upward curve (3) may have a positioning element, foam backing, adhesive or spring mechanism to maintain constant pressure or contact with the EMD (12) to ensure proper measurement during operation. Other examples of the EMD (12) located at or around the fingertip (14c) include a thermal sensor that detects increased temperature of the limb to determine infection, a pulse/oxygen saturation meter that determines blood oxygen concentration to the affected limb, a moisture detection sensor/device that detects active bleed or drainage, a blood coagulation meter that detects blood values to monitor anticoagulant drug therapies, a cardiac output measurement device, an arterial blood gas meter and hemoglobin meter, a glucose meter that detects blood sugar levels, an electrophysiology meter, a micro-assay for blood lab values and a Doppler device that detects blood flow around the affected joint and blood pressure and aberrant blood flow that may signal deep vein thrombosis in the arm. Further examples include a drug delivery device to provide medication through methods such as electricity, i.e., iontophoresis, trans-dermal medication, intramuscular or sub-dermal medication through a needle, a cooling unit that provides circulation of refrigerant or water to decrease inflammation and smart electrodes embedded within the housing or attached to skin. The drug delivery device may provide pain management through transcutaneous electric stimulation, biofeedback, galvanic current, direct current, or indirect current to promote wound healing and/or bipolar current for delivery of drugs using iontophoresis model.

The monopartite housing (2) may be overmolded onto one or more EMDs (12). The housing (2) may have a plurality of interface features that the thumb or other digits can manipulate in an ergonomic fashion to control one or a system of EMDs (12) or an active stylus technology.

One example of the EMD (12) is a radio-frequency identification, or RFID, that uses radio-frequency electromagnetic fields to transfer information. The RFID may provide automatic identification and positional tracking. The RFID may have a battery, be powered by electromagnetic fields, or use a local power source and emit radio waves. The RFID may be either passive, active or battery assisted passive.

The EMDs (12) may be configured within the splint system (1) to be a computer interface device to allow a user to interact with a virtual environment, computer assisted robotic technology, augmented reality system, and/or video game system. The EMD (12) may be a haptic sensor or actuator technology, gesture recognition technology, biometric fingerprint scanner, Apple® Touch Skin, photosensitive material, laser pointer, or measurement device. In another embodiment, the EMD (12) may allow a user to point within or manipulate a volumetric display.

In one embodiment, the housing (2) has a specialized mounting form to accommodate and protect a vibratory haptic device and direct the haptic stimulus towards the users' finger pad (14b) while insulating the stimulus from the rest of the housing (2). A piezoelectric haptic device or piezo strip may also be mounted within the housing (2). An injection-moldable resin with vibration absorbing, electrically insulative or electromagnetic interference, or EMI, shielding properties may be used. Multiple housings (2) may be worn on up to 5 fingers of each hand to allow the user to receive haptic feedback while participating in an augmented reality or virtual reality environment.

In one embodiment, the RFID is embedded throughout the splint system (1) to provide girth measurements. The RFID may interact with a gyroscope and accelerometer to calculate different physical measurements of total limb, joint and total body of a wearer and be incorporated into a fall detection system. This is useful for monitoring patients' movement after surgery and detecting aberrant forces on the splint system (1), such as falls and movements outside safe ranges for surgical grafts or fixations.

With the RFID, a user may be identified by proximity to a touch screen or display in retail, learning, medical, convention or gaming environment. The RFID may also allow physical location to be identified by a RFID scanner system. A user may then opt-in or opt-out of the system based on user preference. The RFID may be used to purchase or place items in a virtual shopping cart through a RFID scanner system in retail setting.

With one or multiple RFID's within a system of splints (1), a user may measure some value of length, temperature, or other physical property using positioning or other physical information data with respect to one another and or an electronic device.

In one embodiment, the splint system (1) may be embedded with a RFID and or correlated magnets and could be part of a security system to gain input access to a computer device or other electronic device. With correlated magnets, the splint (1) may be attached to a personal digital assistant, or PDA, tablet or phone in a predetermined configuration.

In one embodiment, the splint (1) with the RFID and/or correlated magnets is part of a security system to allow a user to unlock the trigger mechanism of a firearm or weapons system. In one embodiment, the splint system (1) is used to protect finger(s) when using weapons. In another embodiment, the splint system (1) is used on the trigger finger or other finger to improve touch or increase friction and decrease vibration or recoil forces and reduce repetitive strain injury while shooting.

The splint system (1) may be configured to include a guitar pick element for use with a musical instrument and/or to protect the finger while playing a musical instrument.

All of the above systems may be monitored or controlled by any electronic device, including a computer device, the splint system (1), or radio frequency, light wave or electromagnetic communication to a server, cloud, and/or nursing station for dissemination and storage so that appropriate action may be taken to ensure user safety, compliance, and/or medical intervention.

A user may operate the splint system (1) by various different steps. First, a user applies the splint system (1) to engage the circumferentially adjustable structures (24) including the wings (6) and (7) and/or ribbing, or ribs, (22) with a portion the finger (14). Then the user positions the upward curve (3) of the splint system (1) near the distal fingertip (14c) such that the upward curve (3) is touching the fingertip (14c) or is within a small distance between the fingertip (14c) and the upward curve (3) of the housing (2).

To activate the EMD (12) embodied in the housing (2), the user may do one of the following: use a gesture of the finger, hand, wrist or arm; manually operate an electromechanical switch located on the housing (2); use a peripheral device to command the EMD (12) with electromagnetic waves; or use a sensor electrode contiguous with the housing (2) to passively or actively sense vibration, sound, movement, chemical substances, biological substances, electromagnetic changes, or physiological changes. Once the EMD (12) is activated, the EMD (12) may send sensor data to a peripheral device using electromagnetic waves or process data with a central processing unit, or CPU, located on the housing (2). A user may also respond to an electromechanical stimulus provided by the EMD (12), such as a haptic actuator or piezoelectric strip, to guide finger movement or carry out an action.

In one variant of this method, the splint system (1) may be activated before donning the housing (2) on the finger.

In one embodiment, the splint system (1) is configured to approximate the tissue surrounding the proximal interphalangeal joint for users with rheumatoid arthritis. In another embodiment, the splint system (1) is configured with a hand/wrist splint to provide proximal support to treat bones, joints, nerves, tendons and soft tissues of the hand, wrist forearm complex.

A rigid or semi rigid plastic point or protuberance or point-shaped stylus or pointer may activate a projective capacitive screen, but not a capacitive sensing screen. Capacitive sensing pointing devices often require a 3-4 mm diameter surface contouring footprint or capacitive contact surface area (15) to be capacitively sensed by the touch screen processor. A hollow conductive rubber tip or foam may activate capacitive sensing touch screen technology. The rubber and foam tip deform on contact, meeting a minimum capacitive surface area (15) about 3-4 mm. A user's natural electrical charge may be conducted through the 3-4 mm surface area of the fingertip (14c) to activate the capacitive sensing screen. The soft tissue structure of the fingertip (14c) is suited to contour to the glass capacitive touch screen.

FIG. 3 shows another embodiment of a splint system (1) with a housing (2) and upward curve (3) with a formation of tines (16) that are preferably made of an electro-conductively doped thermoplastic, thermoplastic elastomer or 3D printed material. There may be any number of tines (16), but preferably, there are two to thirty tines (16). Two sets of winged structures (6) and (7) ergonomically secure the housing (2) to the distal phalange. A functional negative space (11) is created through the straight pull injection molding process. An EMD (12) may embody the functional negative space (11). The surface resistivity of the material is less than 1.0×10⁶ Ohm/square to conduct user's resting electric charge to the touchscreen (18) through the tines (16). The negative space (12) under the distal set of wings (7) may be used to position a small battery to act as a source of electrons or a metal form to act as an electron reservoir, like copper. A battery or metal form may be embedded or fixated to the housing (2) to allow operation when fingers are gloved with an insulative material that blocks the user's resting static charge from flowing through the dielectric housing (2) material. A small, low volt battery will be sufficient to charge the housing (2) for temporary use on a capacitive touch screen. The housing (2) may be used to house an artificial fingerprint technology to allow a user to have a unique identifier to be read by a scanner or computer system. In one embodiment, a quick response code may be applied to the housing (2) to identify the user. In one embodiment, the monopartite splint (1) or finger pad protector with distal interphalangeal collateral ligament support wings (3) may be used as inserts into a gloves' digits to provide support and protection for sports or occupational activities or overmolded onto a glove. As glove inserts, the housing (2) may be reinforced by a stiff material to provide more shock absorption and structural support. In one embodiment, the housing (2) and its various configurations may be used as a bandage or dressing retention device. The splint system (1) with tines (16) is preferably manufactured with the straight pull injection molding technique or with a 3D printing process.

FIGS. 4 and 5 show a side view of a cross section of finger anatomy (17) and one tine structure (16) above a cross section of a touch screen (18). The upward curve (3) formation of tines (16) is designed to activate the screen (18) when the surface of two or more semi-flexible tines (16) deform to the screen (18) and meet the minimum capacitive surface area (15) requirement of the capacitive sensing technology. The soft tissue (19) of the distal fingertip (14c) and finger pad (14b) is a compliant surface supported by the firm bone of the distal phalange (5) and fingernail (20). The upward curve (3) is positioned over the compliant soft tissue (19) supported by bone of the distal phalange (5) improves the surface deforming capacity of the tines (16) and requires minimal force or pressure applied by the user to achieve response from the touchscreen (18).

In FIG. 5, the deformation (21) of one tine (16) is shown between the rigid touch screen (18) and the soft finger pad tissue (19) buttressed by the non-compliant bone of the distal phalange (5). This touchscreen/tine/soft tissue interaction deforms the tine (16), which increases the surface area of the tine (16) on the touchscreen (18), helping to meet capacitive surface area (15) when working in conjunction with an adjacent tine (16) on the upward curve (3). The width of the tine (16) is preferably, about 1.65 mm to 2.0828 mm with a space between tines (16) that is, preferably 0.70 mm to 1.27 mm. The thickness of the tine (16) is preferably about 1.5 mm to 1.6 mm. Measurement of tine width, space between tines (16) and tine thickness will change with choice of materials, advances in molding design and technology, including 3D printing technology. The tines (16) will reduce the tap size to about 3-4 mm similar to that of a hand held stylus, providing increased accuracy and less taps. Coordination may be intuitive as the interface device's surface mimics the natural shape of the finger. The anatomical shape of the splint housing (2) will allow the user the ability to type on a keyboard with increased accuracy. Users with disabilities, including pain, coordination, vision loss or sensory loss, may use their fingers with improved accuracy with touchscreen, flat keyboards or other electronic technologies. Touchscreen users often encounter the parallax problem. It results in user interface errors from the difficulty of targeting the screen from an angle under a layer of glass. The splint system (1) extends these fingertip areas just enough, i.e., about 1.5-1.6 mm, to diminish the inaccuracies caused by the parallax problem.

FIG. 6 shows a frontal view of an upward curve (3) formation of tines (16) that may be suited to meet the minimum capacitive sensing surface area (15) from any angle of user approach. The fingertip anatomy (17) is slightly disengaged making contact with a see-through touchscreen (18). From this perspective, the individual tines (16d), (16e) and (16f) move upward and backward on contact with the touchscreen (18). In this embodiment, the minimal capacitive surface area (15) is met by the 3 tines (16d), (16e) and (16f), which are capacitively sensed. The user may swipe or tap from any position and activate the capacitive touchscreen (18) with a shock absorbing quality.

FIG. 7 shows an inferior upward curve (3) of tines (16) next to a cross section of a touchscreen (18). The tines (16a)-(16i) are in a non-deformed state. The tines (16a)-(16i) contact the touch screen (18) selectively deforming tines (16f) and (16g) and a small portion of tine (16h). This contact area is enough to meet the minimum capacitive surface area (15) to activate the capacitive sensing screen (18).

The tines (16) may be injection-molded and adhered to a non-electrically insulated glove with a conductive adhesive to provide better dexterity to the fingertip (14c) on a capacitive sensing touch screen (18) for a gloved user. The tines (16) may be overmolded onto another surface. The tines (16) may be sprayed or coated in a conductive material onto a glove's electroconductive fabric fingertip to improve user accuracy and performance on the touchscreen (18). The tines (16) may be attached to or configured with a shaft and a backing element for use as a mono- or multipartite injection-molded capacitive stylus. Two opposing upward curves (3) or intertwined upward curves (3) of tines (16) may also be configured to a shaft for use as a stylus.

FIG. 8 shows a cross section of fingertip anatomy (17) slightly disengaged from the tine (16) contacting a cross section of a capacitive touchscreen (18). In this unsupported or non-anatomical tissue backed position, the upward curve (3) flexes backward on contact with the touchscreen (18) demonstrating a spring action caused by the thermoplastic or other resins' material structural qualities. From a transverse perspective, the curved array of tines (16) positioned on the upward curve (3) will engage and flex backwards from the force of the finger directed towards the touchscreen (18). When enough tines (16) contact (about 2-3), the minimum capacitive surface area (15) is met and the tap is capacitively sensed. The user will appreciate a greater shock absorbing capacity of the tines (16) in this disengaged position.

The user will also appreciate better visibility of pixel formations on the screen from different viewing angles because the tines (16) are less view obstructive than that of a solid surface. The tines (16) may allow for better ventilation for the distal finger preventing the buildup of humidity, which may allow the housing (2) to slip off the finger during use. The width and density of the tines (16) may be adjusted to keep pace with changes in touchscreen technology. The material properties, such as durometer and coefficient of friction, of the tines (16) and housing (2) may be adjusted to provide different levels of protection, shock absorption and feel for the user. From a safety profile, the tines (16) on the upward curve (3) significantly reduce the hazard of choking if swallowed by a child because air will flow through the tines (16) if obstructing an airway. An EMD (12), such as a haptic device, may embody the negative space (11) proximal to the tines (16).

In another variation of the splint system (1), the upward curve (3) of tines (16) may be molded or 3D printed to have a perpendicular or diagonal cross support to make a lattice shape. The upward curve (3) may also be molded or 3D printed in triangular shape sections. The upward curve (3) may also be shaped to include a formation of ruffles or pleats in any direction. The upward curve (3) may also have different finishes/textures on the outward facing curve or tines to impart a friction quality during use on the touchscreen (18) or to add utility to occupational tasks.

FIG. 9 shows the splint system (1) with a monopartite housing (2) having a longitudinal housing base (4) and ribbing (22). Features may include winged elements (23) to accommodate the middle phalangeal joint, middle phalange and soft tissue expansion during use and resilient ribbed sections (22) to accommodate the various anthropomorphic circumferential cross sections along the finger. In this embodiment, the ribbing (22) and winged elements (23) are the circumferentially adjustable structures (24). Ribbed sections (22) may be located proximal and distal to the distal interphalangeal joint line and in front of the protruding base of the distal phalange to provide anthropomorphic fit. Ribbing (22) is of suitable anthropomorphic length, shape, width and pattern to secure the housing (2) to the finger at an angle or arcuate form, depending upon user injury. Individual ribs (22) may be cut off by a user to accommodate a deformity of the joint or the finger. An EMD (12) may embody the splint system (1) under the palmar surface (14a) and along the upward curve (3). The sets of ribbing (22) may become concentrically larger from the distal base (4) to proximal base to accommodate the conical anatomy of the finger and to enable a cam molding method of injection molding. The ribbing (22) crosses midline to allow expansion as a finger is engaged in the splint housing (2). As the ribbing (22) expands, they still cover the dorsal surface of the finger providing an improved means of securement to larger girth digits. The width of the ribs (22) is preferably in a range of 0.5 to 1.5 mm. In one example, the ribs (22) are 1 mm. The thickness of the ribs (22) is preferably about 0.25 to 1.0 mm. In one examples, the thickness is about 0.75 mm. Thickness and width may vary with selection of thermoplastic and other resins. The ribbing (22) is a material that will allow the user to quickly don and doff the housing (2) without the need to use Velcro® or other means of securement. In one embodiment, eight ribs (22) and a wing structure (23) provide the functional balance to the splint (1) to prevent pivoting, twisting, axial disengagement and rotation on the finger. Ten points of frictional securing contact along the axial finger may increase the functional stability of the housing (2). A hyperextension force on a tip of the distal housing (2) may be balanced by the winged element (23) arranged at about a 60° angle. A flexion force on a tip of the distal housing (2) tip is balanced by four distal ribs (22). Preferably, the ribs (22) are oval. The oval shape of the ribs (22) provides a stabilizing force on the digit for abduction and adduction forces to the distal fingertip (14c). The oval formation serves to center the finger on the base (4). The position and width of the space between the two sets of ribs (22) is ideally located for the distal phalange. The two sets of ribbing (22) may splint the distal interphalangeal joint during activities of daily living and touchscreen use. Stabilization of the distal interphalangeal soft tissue and joint may allow an inflamed joint to rest. The entire splint system (1) with ribbing (22) may reduce the axial compression of the distal interphalangeal joint and soft tissue by dispersing the force along the base (4) and ten ribbing (22) contact points into proximal more stable joints of the finger.

The housing (2) comprises a length suitable to fit the anthropomorphic length of a finger with an upward curve (3) to meet the palmar surface (14a). The outer face (25) of this curve (3) may have a geometric or non-geometric surface (26), as shown in FIG. 9A. The pattern of the surface (26) may be section of a geodesic sphere, a dimple texture or some other texture that will allow the fingertip (14c) to activate the touchscreen (18) in discrete areas through pressure applied through housing (2) to simulate a fingertip (14c). The texture of the outer face (25) will reduce the coefficient of friction by decreasing the surface area coming into contact with a glass surface. A dual density of material, such as thermoplastic elastomer or elastomer overmolded onto a thermoplastic resin, will allow protection of finger anatomy with a less compliant material while a compliant outer material will reduce impact force. The splint system (1) may also have a plurality of ventilation holes (not shown) for user comfort. The inner finger facing surface (27), including the inner facing ribs sections (22), may have a surface texture that provides increased friction on the finger as the splint is manipulated. In one embodiment, the distal upward curve (3) is removed to allow the distal edge of the finger pad (14b) to protrude. The splint system (1) with ribbing (22) is preferably manufactured with a cam injection molding technique or through 3D printing.

FIG. 10 shows an alternative embodiment of the splint system (1) that has four alternating ribs (22) that cross the longitudinal axis (27) of the splint housing (2). The ribs (22) may be configured as one set of four ribs (22) and a pair of winged elements (23). The longitudinal base (4) may be configured to a length to protect the distal finger pad (14b) and some of the soft tissue of the distal interphalangeal joint complex, including the collateral ligaments and the volar plate with the winged elements (23) for some occupational activities. FIG. 10a shows an exploded view of the outer face (25) with a geodesic sphere patterned surface (26).

The housing (2) may be made of injection-molded or 3D printed materials selected for specific qualities based on the function of splint system (1). A splint system(1) designed for use with the touchscreen (18) may be made of a thermoplastic, elastomer, and thermoplastic elastomer doped with a conductive additive. Materials used for the splint system (1) embodying the EMD (12) may be engineered specifically for physical properties and electromagnetic properties.

The housing shape may be also made by metal stamping, thermosetting, overmolding, casting and any current or future 3D printing technology. Thermoplastic elastomer or elastomers may be overmolded onto a pliable or non-pliable metal or other substrate. Thermoplastic elastomers may be overmolded onto the distal palmer section of the housing (2) along the upward curve (3) to provide a compliant touch. A conductive elastomer, thermoplastic elastomer, fabric or felt may be adhered to the housing (2) using a conductive glue. A conductive film with or without a circuit may also be applied to the outer surface or inner finger facing surface depending on the function of the splint (1). The housing (2) may be made of infrared reflective material or have an infrared reflective coating or film

The monopartite splint (1) may be custom fabricated by taking frontal, sagittal and transverse plane and angular measurements at different points of the digits, hand and wrist either manually or with the use of 3-dimensional (3D) scanner, imaging system or other instrument and then have a splint system (1) 3D printed for immediate use by a user reducing the need for a therapist, physician, trainer or nurse to fabricate and customize the splint from many different materials. The housing (2) may be custom fabricated for the finger or incorporated into a hand and/or wrist splint using 3D printing technology from a user's finger, hand and wrist measurements. The housing (2) may be printed using a 3D printer using any current or future 3D printing materials.

In one embodiment, the splint system (1) is configured to approximate the tissue surrounding the proximal interphalangeal joint for users with rheumatoid arthritis. In one embodiment the splint system is (1) is configured with a hand/wrist splint to provide proximal support to treat bones, joints, nerves, tendons and soft tissues of the hand, wrist forearm complex.

The splint system (1) may be useful in many ways. From a medical perspective, the last joint of any finger is very complex. The splint system (1) may be used as a resting distal interphalangeal splint to support and protect a user with an inflamed joint during activities of daily living. The splint system (1) may be used by occupations and professions at risk for repetitive strain and vibration induced injuries of the finger and hand. Finger flexor muscles end at the distal bone, but finger extensor muscles come together to form a soft tissue extensor slip. When this slip is compromised, people develop hammer finger. The volar plate is also susceptible to stretch injury through constant hyperextension of the distal interphalangeal joint. The finger pad (14b) is a soft tissue structure that is thickest under the distal palmar surface (14a). The very tip at the nail is thinner and will thus be less shock absorbent with an axial impact on a touchscreen.

Repetitive strain is a well-known and studied phenomenon occurring in work or other setting through repetitive motions, vibration or impact about bone, nerves, soft tissue and joints causing chronic and often debilitating injury. Digits of the hand should be protected, particularly since they are prone to stress and injury from the dense glass surface of a touchscreen. Chronic sensory input from vibration and impact along their nerve distributions may make individuals more susceptible to repetitive stress injury, nerve damage and possible conditions like Complex Regional Pain Syndrome. The splint system (1) with the winged structures (6) and (7) will require about four sized versions of different circumferences to fit approximately 95% of the adult population based on current anthropometric data. The splint (1) with ribs (22) may require fewer versions due to its greater extensibility of fit. The splint system (1) is adaptable to both small and large girth fingers. This allows individuals with chubby or fat fingers to use small screens and older users with rheumatoid, osteoarthritis and neurological conditions to improve their coordination because their finger tap will be more accurate.

The vibration and impact absorption qualities of the housing (2) and the physical materials may reduce vibration forces transmitted to the user. As a monopartite housing (2) for interacting with a touchscreen (18), the splint system (1) is a disposable device or reusable after disinfecting procedure for use in a medical settings and may decrease the incidence of nosocomial infection. The splint system (1) with tines (16) will allow the user to operate a touchscreen (18) without leaving a fingerprint, thus reducing identity theft. The material of the splint system (1) will reduce the smudge left on screens and reduce the amount of friction caused by the finger gliding over glass screens. People who sweat or have hyperhidrosis will be able to use their touchscreens without leaving sweat streaks that visually distort on screen content. People with larger finger girths can more accurately use their touchscreens as the tines (16) reduce the tap size to about 3-4 mm.

People with long nails can wear the splint system (1) in a slightly disengaged position from the fingertip (14c), which allows them to operate the screen from any angle of engagement or finger pitch. Many touchscreen users make targeting errors associated with parallax distortion from the layer of glass overlaying the display screen. The splint system (1) will slightly extend the fingertip (14c) improving the user's ability to target the display screen. The splint system (1) will allow the finger to move freely while wearing the EMD (12). The circumferentially adjustable structures (24) will allow the user to easily don and doff the housing (2) quickly and repeatedly while providing a secure fit. The splint system (1) will provide the support needed for a user's delicate finger anatomy while interacting with various current and future technologies. The splint system (1) may be even used to reinforce the anatomy of the finger to allow the finger to penetrate a material or manipulate a material unsuitable for the human finger.

As discussed above, the splint system (1) may be 3D printed by current or future printing technologies. Currently, medical devices, including externally positioned medical devices, anatomically contoured devices, casts, splints and immobilizers, require a user to don multiple garments, bandage systems, electrodes to undergo therapeutic modalities or therapeutic medical regimens for care of a wound, injury or post-operative site. A method for producing a rapid prototype 3D therapeutic surface (30) is desirable to reduce pain, protect a treatment area, accelerate healing and give a treatment area multiple modalities without application of multiple therapeutic systems. FIG. 11 shows a surface (30) made of 3D printed materials that are used in the fabrication of medical devices, including parts integrated with the medical devices, such as splints, braces, immobilizers, anatomically contoured devices and other external medical devices. A therapeutic surface (30) may be part of a splint, brace, immobilizer or external medical device system like the splint system (1). The therapeutic surface (30) may have a support function based on its material composition or it may be supported by a partially or fully enveloping cast, brace, or immobilizer.

Software scenarios may be used to show the step-by-step functional development of the therapeutic surface (30). A planner may be a person or entity that identifies the treatment area and selects passive treatments, active treatments or monitoring systems to be built into the therapeutic surface (30). A therapeutic surface (30) may require a clinical assessment. A physical exam to examine treatment area or areas or regions may be required for the planner to prescribe the appropriate medical, radiological, electrochemical, biological and thermal modalities. Assessment may incorporate a past medical history, imaging studies, operative reports, labs and other tests.

The method includes generating a 3D point cloud of an anatomical surface of the body region targeted for treatment. A software scenario for creating 3D surface mesh may be formed in 2 ways using volume data (e.g., CT (Computerized Tomography), MRI (Magnetic Resonance Imaging), MicroCT, PET (Positron Emission Tomography) scans or other tests) or image data (e.g., surgical photos, x-rays, bone scans or other tests). Volume data may be visualized using a volume data rendering engine. Volume data marking, measurement and segmentation tools may be used to extract the 3D surface point cloud from volume data. A 3D point cloud is triangulated to obtain the 3D surface mesh. Image data may be visualized using an image viewer. A planner may use image data measurement tools to obtain treatment area information and may create a 3D surface mesh manually using modeling tools.

Using a skin side modification module scenario, the 3D surface mesh is modified with various manipulation tools to conform a smooth 3D skin side mesh using the generic modeling tools used for cutting, rounding, smoothing, deleting, and extension.

A template skeleton mesh may be either selected from a database or generated using the 3D skin side mesh properties. 3D skin side mesh analyzing tools may be used to obtain surface parameters. According to analysis, the planner may select a pre-configured template skeleton mesh from a database or the planner may use a template skeleton generation module and generate skeleton mesh using surface parameters. The therapeutic surface template skeleton mesh may be obtained by merging the template skeleton with the 3D skin side mesh.

The software may have a general modeling module scenario to apply basic 3D modeling operations located in a toolbar. Operations may include: hole drilling tools to allow ventilation, mesh scooping tools to model cutouts, mesh extruding tools, mesh smoothing tools, tools to model force/pressure/traction, mesh Boolean operations for merging operations and mesh cutting tools.

The method also includes selecting active and passive treatments that will direct the layout and design of the therapeutic surface (30). Planners may also select monitoring systems to include in the therapeutic surface (30). A planner may drag and drop the active and passive treatments to the therapeutic treatment surface template skeleton treatment area or position with x, y, and z coordinates. In a preferred embodiment, the 3D modeling scenario consists of several modes for quick modeling and integration of a system of features of holes (31), conduits (32), channels (33), reservoirs (34), 3D printed electronic circuits (35), textures (36), geometric forms (37), non-geometric forms (38), biomimetic structures (39), 3D printed electrodes (40), 3D printed coatings (41), 3D printed optics (43), cavities (44) and/or 3D materials layered or positioned according to material function within the surface (30). Each mode may have special visualization properties, such as window layout and view settings, and a specific toolbar with mode specific modeling tools. Specific modeling tools may include a general modeling mode, electromechanical device (10) insertion mode, serial joint casting mode, dynamic splinting mode, body securing mode, material functions mode, joint segmentation mode, external element holder design mode, external element positioning mode, fluid circulator design mode, component positioning module, 3D modeling module and a special holder design mode. In a preferred embodiment, the software will build up the thickness of the treatment surface (30) as required by selected active and passive treatments parameters.

The 3D printed therapeutic surface (30) and the system of features (31)-(44) may work independently or in conjunction with an electromechanical device (10) contiguous with the 3D printed surface (30). The electromechanical devices (10) may have 3D printed ports (51), 3D printed valves and 3D printed electromechanical device interface features. The 3D printed features may be contiguous with the exterior facing (47) and inner skin facing (42) of the therapeutic surface (30). The computer software 3D modeling tools may position features and materials within the surface (30) to maximize the delivery of medical, thermal, electrochemical, radiological, biological or light based therapies to the treatment area using algorithms and/or planner input.

An electromechanical device insertion scenario may comprise the following. The planner filters out the electromechanical devices (10) according to their functionality and according to where they will be inserted (skin side face (42) or exterior face (47)). The filtering component affects the available passive components for that electromechanical device (10). The selected passive components, such as conduits (32), channels (33), cavities (44), and reservoirs (34), may be modified within the allowed parameters for a selected electromechanical device (10). A conduit (32), channel (33), cavity (44) or reservoir (34) may be sized and insulated appropriately to meet the technical requirements of the electromechanical device (10). Different electromechanical devices (10) may have different required proprietary and nonproprietary configurations to be accounted for in layout and design of the therapeutic surface (30). The planner may enable the delivery maximization simulator, which visually guides the planner to do the necessary modifications to maximize the efficiency in delivery of active and passive systems. The supportive stay or mount of each electromechanical device (10) may be updated according to filtering where the planner may select from the passive components toolbar. 3D printed circuits (35) may be configured by the electromechanical device insertion scenario for safety and insulative value.

A material functions module software scenario may manage the layout and deposition of different 3D printed materials. The planner may use the material functions toolbar to manage the 3D material related operations. The planner may select 3D materials according to physical, thermal, antibacterial/antifungal, chemical, electromagnetic and biological properties. 3D materials may be applied in several ways: directly altering the selected therapeutic surface skeleton mesh faces layered on top of the selected therapeutic skeleton mesh faces, externally covering for insulation purposes, internally coating to improve fluid dynamics, such as in channels (33), internally coating for skin contact points or to provide padding structure, or internally depositing to absorb mechanical or electromagnetic energy forces. 3D printed materials may be positioned for strength, flexibility or function within the 3D printed therapeutic surface (30) by the planner. 3D printed material functions may include padding, antibacterial/antifungal properties, electrochemical insulation/conduction, thermal insulation/conduction and flexibility and rigidity. 3D printed materials may provide properties similar to various metal and metal alloys, various plastic and elastomeric resins, ceramics, carbon fiber based and nanomaterials. Certain 3D materials may be deposited within the 3D printed surface (30) similarly to trabecular lines (45) in bones to absorb forces along the surface axially and torsionally to protect the surface covered body part. Other 3D patterns, regular or irregular, such as lattices or honeycomb formations, may add strength or other physical properties. This 3D material deposition will reduce the weight of the splint, brace, immobilizer, and medical device and increase the strength. 3D materials may be deposited in a helical or coiled shape (46) to mimic connective tissue. 3D materials that are insulative thermally and electrochemically may be positioned around 3D printed channels (33), reservoirs (34) and conduits (32) to prevent thermal changes or electronic signal deterioration. Certain 3D materials that have antifungal, antibacterial properties and drug-like properties, such as silver or 3D printable drugs or UV cured drugs, may be layered or deposited as a coating (41) on the skin contact portion of the surface or throughout the physical features of the therapeutic surface (30). Charged coatings (41) may also be deposited to draw certain ions to the coating (41) or repel certain ions from the coating (41). The coatings (41) may be able to change the chemical property of the wound to promote healing. Coatings (41) may be selectively positioned to perform a certain function on the surface (30), padding (48) or membrane (50). These coatings (41) will aid healing of wounds or burns or provide growth to engineered tissues. Certain 3D materials that have antifungal, antibacterial and drug-like properties, such as silver or 3D printable drugs or UV cured drugs, may be layered or deposited as a coating (41) on the skin contact portion of the surface or throughout the physical features of the therapeutic surface (30).

A body securing module scenario may allow for the therapeutic surface (30) to be secured to the body. The planner may use a body securing toolbar to select the hardware components and 3D printed geometric components (37) and non-geometric components (38) for securing. Body securing components may be filtered according to the body part on which they will be secured. The planner may merge the selected components to therapeutic surface skeleton mesh. Geometric forms (37) or non-geometric forms (38) may be externally and internally included for attachment of hardware or positioned supportive stays. The therapeutic surface (30) may be monopartite or segmented into a multipartite surface. The therapeutic surface (30) may have adjustable sections and preconfigured areas for tissue swelling to better accommodate for limb volume changes. Segments of the therapeutic surface (30) may communicate through connector modules to allow continuity of therapeutic modalities throughout a multipartite therapeutic surface (30). A joint segmentation module scenario will allow the planner to select a joint segmentation toolbar to segment the therapeutic surface skeleton mesh into several parts. The planner may use the appropriate mesh cutting tools from the toolbar. The planner may select and merge the appropriate joint holder or connector modules on therapeutic surface skeleton mesh.

A 3D printed surface (30) may be used to serially cast a joint in different positions while delivering modalities to reduce tissue contractures, reduce pain and increase tissue extensibility. To accomplish this goal, the software may have a serial joint casting module scenario. The planner may use the serial joint casting toolbar to adjust the therapeutic surface template skeleton mesh. A serial joint casting toolbar may contain surface modeling tools to increase alignment, reduce tissue contracture, reduce pain, and increase tissue extensibility. An alignment indicator may be used to see the alignment values in real time and do the therapeutic surface (30) modifications accordingly. The 3D printed therapeutic surface (30) may also be configured to apply force/pressure or reduce pressure/force or provide a traction force through use of the shape or hardware of the surface (30) to a fractured bone to improve alignment and prevent surgical open reduction internal fixation. Hardware, such as springs, pulleys, hinges, expansion bolts or elastomeric bands, may be added through attachment to geometric forms (37) and non-geometric forms (38) arising from the surface (30) at different intervals to provide dynamic splinting. A planner may use the dynamic splinting module scenario to simulate therapeutic surface design. The planner may select from the dynamic splinting toolbar the hardware components that the planner wants to simulate on therapeutic surface skeleton mesh. The planner may then select the necessary geometric components (37) or non-geometric components (38) for a corresponding dynamic splinting component and merge it onto the skeleton mesh of the therapeutic surface (30).

A fluid circulator design module scenario may allow the planner to select the fluid circulator design toolbar to model channels (33), reservoirs (34), bladders (49) or membranes (50). Channels (33) and reservoirs (34) may be shaped and positioned to improve fluid dynamics. 3D printed coatings (41) may be positioned within fluid based features to provide antibacterial/antifungal properties and improve fluid dynamics. Channels (33) throughout the 3D printed surface (30) may allow coolant to be circulated by a therapeutic electromechanical device (10) to reduce inflammation or warming fluid to increase circulation. Although FIG. 11 shows these fluid/gas circulation features (32), (33), and (34) in a cross-sectional view, it may be appreciated that the channels (33), conduits (32) and reservoirs (34) may run in all directions between the inner surface (42) and external surface (43). Conduits (32), channels (33) and reservoirs (34) may be used to deliver or circulate blood products or liquid substances to the area of injury. The internal surface (42) of the 3D printed therapeutic treatment surface (30) may have geometric forms (37) and non-geometric forms (38) and cavities (44) for inclusion of externally elements, such as electrodes, membranes, dressings, bandages, wound care elements and sensor electrodes. An external element holder design module scenario may allow a planner to select from the external element holder design toolbar to create the holders. The planner may choose the appropriate external holder element (electrodes, membranes, dressings, bandages, wound care elements, sensor electrodes and other elements) and load its 3D file from the database. Using movement handles, the planner may position the holder on the desired place on the therapeutic skeleton mesh and merge it to complete the holder integration. The 3D printed surface (30) may be segmented at different joints to provide directional flexibility if necessary to heal an injury. Cutouts may also be added to prevent compression on healing tissues or bony prominences. A plurality of holes (31) may be positioned throughout the 3D printed surface (30) to allow ventilation of tissue from the inner skin side surface (42) to the external surface (43). A distribution of ventilation holes (31) maybe spread throughout the therapeutic surface (30) to allow circulation of air from the skin side (42) to the external side (47). The ventilation holes (31) may interact with a blower or negative pressure vacuum to circulate fresh air under the treatment surface (30). The ventilation holes (31) may be used to circulate and remove sterilization fluid or gas along the skin surface by a disinfection purposed pump system.

Using a 3-modeling module scenario, geometric forms (37) or non-geometric forms (38), and patterns and textures (36) may be printed on the interior, skin facing surface (42) to decrease hypersensitivity, increase vascularity, increase skin extensibility and reduce scar formation. These geometric forms (37) and non-geometric forms (38) may work in conjunction with an electromechanical device (10) to stretch or stimulate scar tissue breakup or promote circulation. These geometric forms (37), non-geometric forms (38), patterns and textures (36) on the inner skin facing surface (42) may work in conjunction with an electromechanical device (10) to channel gases or fluids along the skin facing surface to obtain a therapeutic effect.

A component positioning module scenario will allow the planner to select from a component positioning toolbar to position and modify the therapeutic surface skeleton mesh accordingly. The planner may select the 3D mesh file of the desired external or internal component (e.g., intrinsic/extrinsic circuit, intrinsic/extrinsic sensor, intrinsic/extrinsic actuator, haptic device, optics, needle, plunger, window). Using the positioning handles, the planner may position the elements on the therapeutic skeleton mesh. If needed, the therapeutic surface (30) is automatically modified (scooped, smoothed, or drilled) to be able to make the component fitting possible. A spacer function will allow the necessary spacing between components. Intrinsic 3D printed circuits (35), extrinsic electronic circuits, intrinsic 3D printed sensors or actuator electrodes (40) and extrinsic sensor or actuator electrodes may be positioned throughout a 3D printed surface (30) to deliver medicine through iontophoresis, provide wound healing through galvanic or other current, stimulate the formation of bone, provide pain relief through transcutaneous electrical stimulation, cause muscle contraction through neuromuscular electric stimulation and sense physical, chemical or biological processes. Electromechanical haptic devices may be positioned throughout the therapeutic surface (30) to provide pain relief and decrease sensitivity of injured tissue or treatment area. The skin facing surface (42) shows two 3D printed electrodes (40) that may deliver a therapeutic electrical modality or provide a sensor function to monitor a wound or injury. 3D printed optics (43) made of 3D printed translucent materials may be configured in the surface (30) to work in conjunction with an electromechanical device (10) to provide a light based therapy or monitor a wound without removal of the 3D printed surface (30). 3D optics may interface with a microscope for pathology examination. A 3D printed surface (30) may have a reservoir (34) to deliver medication to an injury or store drainage from a wound. Conduits (32) may allow the placement of wiring, diodes or tubing within the 3D printed surface (30). The 3D printed surface (30) may work independently or in conjunction with a 3D padding structure (48) or extrinsic padding, 3D printed bladder structure (49) or extrinsic bladder or 3D printed membrane (50) or extrinsic membrane. An extrinsic bladder or 3D printed bladder (49) may be used in conjunction with the 3D printed surface (30) to provide a constant or segmental vasopneumatic compression to reduce edema or circulate warming or cooling fluid depending on injury. Extrinsic padding or a custom 3D printed closed cell padding (52) and open cell padding (53) with germ resistant coating (41) may be used in conjunction with the 3D printed surface (30). An extrinsic membrane or custom printed 3D membrane (50) to reduce skin maceration, maintain sterile environment or direct wound drainage may be used in conjunction with the 3D printed surface (30). A non-electromechanical device, such as a needle or plunger, may also be integrated to a therapeutic surface (30) to work in conjunction with a geometric form (37) to deliver medicine or a window to take lab samples/biopsies. A component positioning module scenario may be used to position a 3D syringe geometric formation that may have an internally or externally printed dosing meter. A window element that communicates through the therapeutic surface (30) may be located on a slide track that is printed of 3D materials. The window element may be positioned by the component positioning module scenario to allow the user to access the skin side surface (42) for wound care, biopsy, suture removal and other medical procedures without removal of the therapeutic surface (30).

Special holder's scenarios with appropriate function toolbars may be included for external therapeutic components that require the system of features for integration into the therapeutic surface (30). They include bone stimulator integration, mechanical range of motion device integration, dermatology/plastic surgery therapies integration, infusion pump integration, hyperoxygenation delivery system integration and negative pressure vacuum integration. Other current and future medical technologies may be integrated into the 3D printed surface (30). A bone stimulator is especially necessary in the non-weight bearing bones to promote healing of fractures. A therapeutic surface (30) may allow a vacuum to be generated around the treatment area for healing purposes. A wound vacuum apparatus may also be configured with 3D printed surface (30). A therapeutic surface (30) may be used to target dermatological, cosmetological or plastic surgery therapies to improve skin quality and managed adipose tissue when used in conjunction with an electromechanical device (10). A mechanical range of motion apparatus may also be configured to a 3D printed surface (30). A hyperoxygenation delivery system may be integrated into the 3D printed surface (30) to help the healing of wounds. A portable infusion pump may be integrated to a therapeutic surface (30). A therapeutic surface (30) made of 3D printed materials, such as lead, to block radiation may be configured to allow targeted delivery of radiological therapy while protecting surrounding tissues. A therapeutic surface (30) may be after processed with a radiation blocking coating or material. A therapeutic surface (30) may be used in conjunction with an electromechanical device (10) to control an implanted medical device/system or medical therapy system.

A therapeutic surface (30) may be designed to provide short-term protection and healing aid to subcutaneous tissues in the event that cutaneous tissues need to be removed. An externally positioned or internally implanted tissue growth substrate, artificial formation base or scaffolding to grow engineered tissue may be integrated into an externally positioned therapeutic surface (30) to provide protection and nutrition while new tissue develops. The 3D surface (30) may be printed in sterile conditions to reduce risk of infection.

A finite analysis of therapeutic surface (30) may be performed before printing to address surface and system features' performance with or without the electromechanical device (10). A therapeutic surface (30) may be after-processed with an adhesive to allow temporary bonding to the skin. The therapeutic surface (30) may be after-processed to add finishing elements, extrinsically applied coatings, seals, valves or fittings to allow interaction between the electromechanical device (10) and the therapeutic surface (30). The 3D surface sensors and electromechanical devices (10) may have monitoring systems, including CPUs, memory, power source and wireless communication systems, to externally monitor the patient, devices or treatment area. The 3D printed therapeutic surface (30) may be preconfigured with different systems of features for various injuries or pathologies of body parts in various sizes to be kept as stock on location or for centralized distribution. In a preferred embodiment, the components of the therapeutic surface fabrication system are a CPU, memory storage, a planning station with visual and interface controls, a local or cloud network, a software program and a 3D printer.

The therapeutic surface (30), while presented as flat in FIG. 11, may be shaped to be anatomically contoured to any external anatomical feature as derived from any bodily 3D scans, imaging, photos and surface scans. The therapeutic surface (30) may be used in any field of practice, including human medicine or veterinary medicine. Both the internal skin side (42) and external facing side (47) of the therapeutic surface (30) demonstrate the necessity to build up the thickness of the device, depending on the complexity of the passive and active treatments systems and their required parameters. Thickness of the therapeutic surface (30) may also be built up or thinned out depending on the strength and quality of the 3D materials used. Each therapeutic surface (30) thickness level may vary based on user's need and planner requirements. The 3D modeling software may use a database of 3D models and planner input to optimize design.

Although the invention has been described in detail and with reference to specific embodiments, it will be apparent to one skilled in the art that various changes and modifications can be made without departing from the spirit and scope of the invention. Thus, it is intended that the invention covers the modifications and variations of this invention provided they come within the scope of the appended claims and their equivalents.

What is claimed is:

1. A splint system comprising:
a housing having a base with ends and sides and at least one circumferentially adjustable structure contiguous with the base extending from the sides to a longitudinal axis of the base for fixing the housing to at least one finger.

2. The splint system of claim 1 wherein the base comprises a flat surface along the longitudinal axis.

3. The splint system of claim 1 wherein the base comprises a curved surface centered along the longitudinal axis.

4. The splint system of claim 1 wherein the circumferentially adjustable structure comprises at least one of ribs and wings.

5. The splint system of claim 4 wherein the wings curve inward toward the longitudinal axis

6. The splint system of claim 4 wherein the ribs curve inward and over the longitudinal axis.

7. The splint system of claim 4 wherein end portions of the ribs overlap with each other along the longitudinal axis.

8. The splint system of claim 1 wherein the base comprises an upward curve that extends from one end of the base.

9. The splint system of claim 8 wherein the upward curve comprises a plurality of tines.

10. The splint system of claim 9 wherein the plurality of tines comprise a range of two to thirty tines.

11. The splint system of claim 1 wherein the base comprises a negative space opposing the wings.

12. The splint system of claim 1 wherein the base comprises a negative space opposing the wings and extending distally up to a tip of the upward curve.

13. The splint system of claim 1 further comprising an electromechanical device embodied in the housing.

14. The splint system of claim 13 wherein the electromechanical device comprises at least one of a biological, chemical, thermal, light, mechanical, and electromagnetic function.

15. The splint system of claim 13 wherein the electromechanical device comprises at least one of a nano-electromechanical device and a micro-electromechanical device.

16. The splint system of claim 13 wherein the electromechanical device comprises at least one of RFIDs and correlated magnets.

17. The splint system of claim 1 wherein the housing comprises at least one of a mono-partite housing and a multi-partite housing.

18. A splint system comprising:
a housing having a base with ends and sides and at least one circumferentially adjustable structure contiguous with the base extending from the sides to a longitudinal axis of the base for fixing the housing to a palmar surface of any sized finger;
an upward curve extending from one end of the base; and
an electromechanical device embodied within the housing.

19. A method interfacing with a touchscreen comprising:
providing a splint system with a housing having a base with ends and sides, at least one circumferentially adjustable structure contiguous with the base extending from the sides to a longitudinal axis of the base, and an electromechanical device embodied in the base, one end of the base having an upward curve extending therefrom;
engaging the circumferentially adjustable structures with a portion the at least one finger;
positioning the upward curve near a tip of the at least one finger; and
activating the electromagnetic device.

20. The method of claim 19 wherein the activating comprises at least one of:
gesturing the finger;
manually operating an electromechanical switch located on the housing;
operating a peripheral device to command the electromechanical device with electromagnetic waves; and
using a sensor electrode contiguous with the housing to sense vibration, sound, movement, chemical substances, biological substances, electromagnetic changes, or physiological changes.
